(19) United States
(12) Patent Application Publication
(10) Pub. No.: US 2026/0091224 A1
(43) Pub. Date: Apr. 2, 2026

(54) Flexible Electrode Array

(71) Applicant: Novocure GmbH, Root D4 (CH)

(72) Inventors: Elie Yaacobi, Haifa (IL); Noa Halavee, Tel Aviv (IL)

(21) Appl. No.: 18/902,150

(22) Filed: Sep. 30, 2024

(51) Int. Cl. A61N 1/36; A61N 1/04

(52) U.S. Cl. CPC A61N 1/36002; A61N 1/0476; A61N 1/0484

(57) ABSTRACT

A transducer apparatus for delivering tumor treating fields to a subject's body is disclosed. The transducer apparatus can include an array of electrode elements arranged around a centroid and each electrode element equidistant or approximately equidistant from the centroid. The transducer apparatus can further include a PCB or flex circuit including at least one electrical connector portion. Each respective electrical connector portion of the at least one electrical connector portion can extend between a corresponding pair of adjacent electrode elements of the array of electrode elements. Each electrode element of the array of electrode elements includes a portion most proximal to the centroid of the array, and each electrical connector portion is spaced radially outwardly of the portions of the corresponding pair of adjacent electrode elements most proximal to the centroid of the array.

### FIELD

This application relates to apparatuses for providing Tumor Treating Fields.

### BACKGROUND

Tumor Treating Fields (TTFields) therapy is a proven approach for treating tumors using alternating electric fields at frequencies between 100-500 KHz. The alternating electric fields are induced by transducer apparatuses (e.g., arrays of capacitively coupled electrodes, also called transducer arrays or electrode assemblies) placed on opposite sides of a target location in the subject's body. When an AC voltage is applied between opposing electrode assemblies, an AC current is coupled through the electrode assemblies and into the subject's body.

Proper positioning of electrode arrays relative to each other and a target region (e.g., a tumor) can affect performance of treatment. However, inflexibility of the transducer apparatuses can inhibit placement and adhesion to the subject and may negatively impact comfort in use for the subject. Accordingly, transducer apparatuses with improved flexibility are desirable.

### SUMMARY

Disclosed herein, in one aspect, is a transducer apparatus for delivering tumor treating fields to a subject's body. The transducer apparatus can include an array of electrode elements arranged around a centroid and each electrode element equidistant or approximately equidistant from the centroid. The transducer apparatus can further include a PCB or flex circuit including at least one electrical connector portion. Each respective electrical connector portion of the at least one electrical connector portion can extend between a corresponding pair of adjacent electrode elements of the array of electrode elements. Each electrode element of the array of electrode elements includes a portion most proximal to the centroid of the array, and each electrical connector portion is spaced radially outwardly of the portions of the corresponding pair of adjacent electrode elements most proximal to the centroid of the array.

In another aspect, a transducer apparatus for delivering tumor treating fields to a subject's body comprises a plurality of electrode elements having a centroid and arranged in a circular or oval region around the centroid. The plurality of electrode elements comprise at least a first electrode element and a second electrode element. The transducer apparatus further comprises a printed circuit board (PCB) or flex circuit comprising a plurality of electrical connector portions. Each electrode element of the plurality of electrode elements is connected to at least one adjacent electrode element in the circular or oval region by a respective electrical connector portion of the plurality of electrical connector portions. At least one electrical connector portion of the plurality of electrical connector portions has an alternating-profile extending between the first and second electrode elements.

Also disclosed herein is a method of applying tumor treating fields to a subject's body, the method can include positioning a first transducer apparatus as disclosed herein in a first position at a first location of the subject's body. An electric field can be induced between the first transducer apparatus and a second transducer apparatus located at a second location of the subject's body.

### BRIEF DESCRIPTION OF THE DRAWINGS

FIG. 1 is a top plan view of an exemplary electrode array assembly in accordance with embodiments disclosed herein.

FIG. 2 shows a top plan view of an exemplary electrode array assembly in accordance with embodiments disclosed herein having a portion with an alternating-profile between adjacent electrodes.

FIG. 3 shows a plurality of electrode array assemblies as disclosed herein positioned on a head of a subject.

Various embodiments are described in detail below with reference to the accompanying drawings, wherein like reference numerals represent like elements.

### DETAILED DESCRIPTION

This application describes apparatuses (e.g., exemplary electrode array assemblies and/or treatment assemblies) that can be used, e.g., for delivering TTFields to a subject's body and treating one or more cancers or tumors located in the subject's body.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Implementations illustrated under any heading or in any portion of the disclosure may be combined with implementations illustrated under the same or any other heading or other portion of the disclosure. Any combination of the elements described herein in all possible variations thereof is encompassed by the invention unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Optionally, in some aspects, when values are approximated by use of the antecedents “approximately,” “about,” “substantially,” or “generally,” it is contemplated that values within up to 50%, up to 25%, up to 15%, up to 10%, up to 5%, or up to 1% (above or below) of the particularly stated value or characteristic can be included within the scope of those aspects.

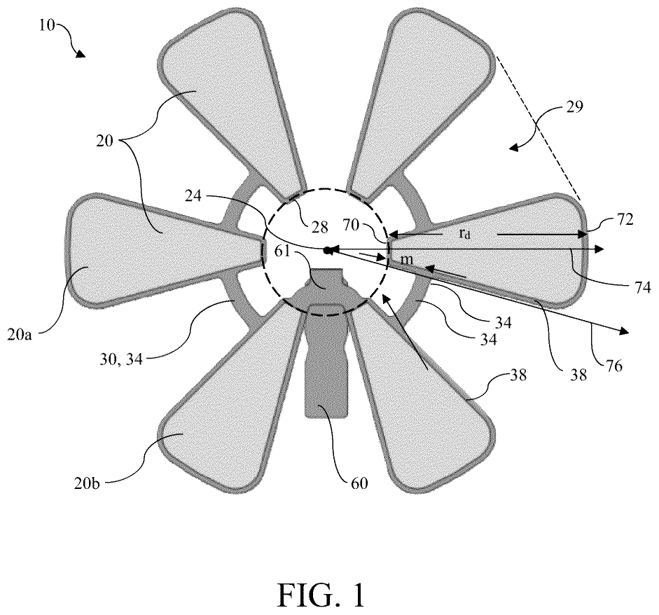

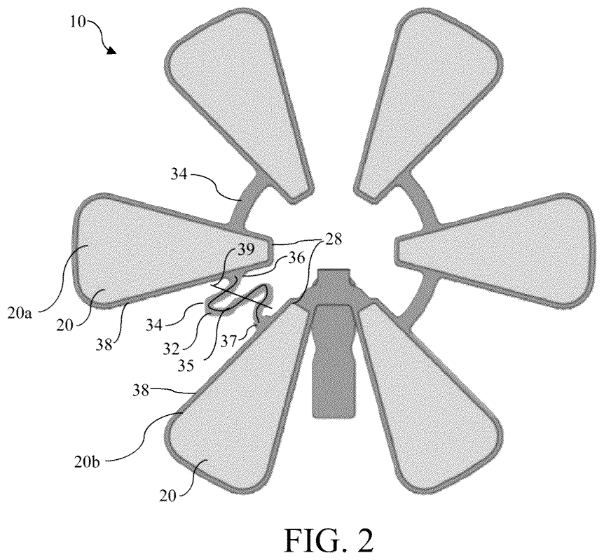

Disclosed herein, in various aspects, and with reference to FIGS. 1-2, is a transducer apparatus 10 for delivering tumor treating fields to a subject's body. The transducer apparatus 10 can comprise an array of electrode elements 20 comprising at least a first electrode element 20a and a second electrode element 20b. The transducer apparatus 10 can further comprise a printed circuit board (PCB) or flex circuit 30. In some aspects, the array of electrode elements 20 can have a centroid 24, which can correspond to the geometric center of the array. The array of electrode elements 20 can comprise a plurality of electrode elements arranged in a circular or oval region around the centroid 24. In some aspects, the electrode elements 20 can extend radially outwardly from the centroid 24. Optionally, the electrode elements 20 can be symmetric about respective axes extending radially from the centroid. For example, the position of the electrode elements about the centroid may be such that the array of electrode elements possess rotational symmetry about the centroid. For example, an array of n electrode elements may have Cn rotational symmetry. The PCB or flex circuit 30 can further comprise a plurality of electrical connector portions 34. Each electrode element 20 of the plurality of electrode elements can be connected to at least one adjacent electrode element in the circular region by a respective electrical connector portion 34. In some aspects, each electrical connector portion 34 can be spaced radially outwardly of the portions of the corresponding pair of adjacent electrode elements most proximal to the centroid of the array.

In some aspects, a plurality of electrical connector portions can be arranged in a circular, substantially circular, oval, or substantially oval profile about the centroid.

Each electrode element 20 of the array of electrode elements can have an end 70 proximal (closest) to the centroid 24, an end 72 distal to (farthest from) the centroid, and a radial dimension, r_{d}, measured between the end proximal to the centroid and the end distal to the centroid. For example, a radial line 74 extending from the centroid 24 can intersect the end 70 of the electrode element proximal to the centroid and the end 72 distal to the centroid. The radial dimension, r_{d}, can be a length along the radial line 74 from the end 70 to the end 72. Optionally, the radial line 74 can bisect the electrode element 20. In some aspects, each electrical connector portion 34 of the plurality of electrical connector portions can connect to each respective electrode element at a location positioned between 10% and 90% of the radial dimension along the radial dimension of each electrode element of the respective pair of electrode elements, said location being measured as a percentage along the radial dimension of the electrode element proceeding from the end proximal to the centroid to the end distal to the centroid. For example, said % of the radial dimension can be a ratio of (a) a measure, m, along a radial line 76 from the centroid 24, where m measures from the radial distance of the end 70 to a point of intersection where the electrical connector 34 meets an edge 38 of the electrode element 20 to (b) the radial dimension, r_{d}. In various aspects, each electrical connector portion 34 of the plurality of electrical connector portions can connect to each respective electrode element at a location positioned between 10% and 90%, or between 15% and 90%, or between 20% and 90%, or between 25% and 90%, or between 50% and 90%, or between 10% and 75%, or between 25% and 75%, or between 50% and 75%, or between 25% and 50%, or between 90% and 100% of the radial dimension along the radial dimension of each electrode element of the respective pair of electrode elements. In additional exemplary aspects, the plurality of electrical connector portions can connect to each respective electrode element at a location positioned between 10% and 75%, or between 10% and 50%, or between 10% and 40%, or between 10% and 30% of the radial dimension along the radial dimension of each electrode element of the respective pair of electrode elements.

In other aspects, each electrical connector portion 34 of the PCB or flex circuit 30 can be spaced radially outwardly of the portions of the corresponding pair of electrode elements most proximal to the centroid 24 of the array. For example, the portion 28 of each electrode element 20 most proximal to the centroid 24 of the array of electrode elements can be spaced from the centroid by a first radius (shown as a dotted line in FIG. 1), and each electrical connector portion is spaced from the centroid by a radius that is at least 15%, or at least 20% greater than the first radius. In further aspects, each electrical connector portion is spaced from the centroid by a radius that is at least 25%, 50%, 100%, 150%, 200%, 300% greater than the first radius. It is contemplated that a greater spacing from the first radius can provide a greater flexibility of the transducer 10. In some aspects, one or more electrical connectors having an alternating-profile 32 (FIG. 2) can also be positioned to couple adjacent electrode elements 20 at the respective positions of the electrode elements radially outwardly from the portions of the electrode elements 28 most proximal to the centroid of the array. In other aspects, one or more electrical connectors having an alternating-profile 32 can be positioned to couple adjacent electrode elements 20 at the respective portions 28 of the electrode elements most proximal to the centroid of the array.

Referring to FIG. 2, in some aspects, at least one electrical connector portion 34 of the plurality of electrical connector portions can have an alternating-profile 32 extending between adjacent electrode elements 20. For example, the alternating-profile can be undulating, serpentine, wavelike, or zig-zagged. In some aspects, the alternating-profile can have a spiral shape. The alternating-profile can permit flexibility of the transducer apparatus and, in particular, permit flexibility between the adjacent electrode elements 20.

In some aspects, the electrical connector portion 34 having an alternating-profile 32 can have a first end 36 and a second end 37. In various aspects, the alternating-profile can have at least two direction changes between the first and second ends 36, 37. In further aspects, the alternating-profile can have at least three, at least four, at least five, or at least six direction changes between the first and second ends 36, 37. In some aspects, the direction change can be defined by a change in concavity (e.g., from a concave profile to a convex profile). In some aspects, the direction change can be defined within a section of the electrical connector portion 34 positioned between respective first and second locations along the length of the electrical connector portion where an imaginary plane 39 intersects the electrical connector at least twice or least three times.

The alternating-profile can have a linear dimension measurement (referred to herein as a “straight length”) between the first and second end measured along a straight line between the first and second ends 36, 37, and can have a curve length measured along a curve 35 on the surface of the alternating-profile centered between opposed side edges 38 of the alternating-profile. In some aspects, the alternating-profile can have a curve length that is at least 10% greater than, at least 20% greater than, at least 30% greater than, at least 40% greater than, at least 50% greater than, or at least 100% greater than the straight length.

In some embodiments, the electrode elements 20 can be non-ceramic dielectric materials positioned over a plurality of flat conductors such as, for example, polymer films disposed over pads on a PCB or flex circuit or over flat pieces of metal. In other embodiments, the electrode elements 20 can be ceramic elements comprising a ceramic dielectric material. In still further aspects, the electrode elements can be free of a dielectric material. For example, the electrode elements 20 can comprise or be a plurality of flat conductors of the PCB or flex circuit 30.

In some aspects, the transducer apparatus 10 can comprise from two to eight electrodes or from two to six electrodes. In various aspects, the transducer apparatus 10 can have exactly three electrodes, exactly four electrodes, exactly five electrodes, exactly six electrodes, or exactly eight electrodes. In one aspect, the electrode elements 20 of the plurality of electrode elements can be equidistant or approximately equidistant from the centroid 24.

In exemplary aspects, each electrode element 20 of the plurality of electrode elements can define a taper toward the centroid 24. In some aspects, the electrode elements can have the same or substantially the same shape and the same or substantially the same area. In exemplary aspects, the transducer apparatus 10 can have a void space 29 between adjacent electrode elements that has an area approximately equal to the area of the electrode elements.

In some aspects, the transducer apparatus 10 can comprise a pair of adjacent electrode elements that are not connected by an electrical connector portion 34 extending therebetween. For example, optionally, the transducer apparatus 10 can have n electrode elements and n-1 electrical connector portions 34. For example, the transducer apparatus 10 can have six electrode elements (n=6) and five electrical connector portions 34. In other examples, the number of electrode elements n may be five, or n may be four, or n many be three. In additional aspects, the transducer apparatus can have n-2 electrical connector portions 34, and a bridging connector 61 extending between two of the electrode elements. The bridging connector can further be configured to couple to an AC current source. For example, the bridging connector 61 can further comprise an electrical connector 60. Optionally, the bridging connector 61 can couple to the electrode elements 20 at or near the respective ends 70 proximal to the centroid 24. In other aspects, the bridging connector 61 can be spaced radially from the ends 70 of the electrode elements 20. By way of example, in these aspects, the number of electrode elements may be six (n=6). For example, the transducer apparatus 10 can have six electrode elements, four electrical connector portions 34, and a bridging connector 61. In other examples, the number of electrode elements may be five (n=5), or may be four (n=4), or may be three (n=3).

Each respective electrical connector portion 34 of the plurality of electrical connector portions can extend between a corresponding pair of adjacent electrode elements 20 of the array of electrode elements.

In some optional aspects, the plurality of electrical connector portions 34 can be arranged in a circular profile about the centroid 24. Optionally, each electrical connector portion 34 can be curved, having a radius that is equal to, or approximately equal to, the radial spacing of the electrical connector portions 34 from the centroid.

In exemplary aspects, a transducer apparatus 10 for delivering tumor treating fields to a subject's body can comprise an array of electrode elements 20 arranged around a centroid 24 (of the array), with each electrode element being equidistant or approximately equidistant from the centroid. The transducer apparatus 10 can further comprise a PCB or flex circuit 30 comprising at least one electrical connector portion 34. Each respective electrical connector portion 34 of the at least one electrical connector portion can extend between a corresponding pair of adjacent electrode elements 20 of the array of electrode elements. Each electrode element 20 of the array of electrode elements can comprise a portion 28 most proximal to the centroid 24 of the array. Each electrical connector portion 34 can be spaced radially outwardly of the portions of the corresponding pair of adjacent electrode elements most proximal to the centroid of the array.

The transducer apparatus 10 can further comprise an electrical connector 60 for coupling to a current/voltage generator (not shown).

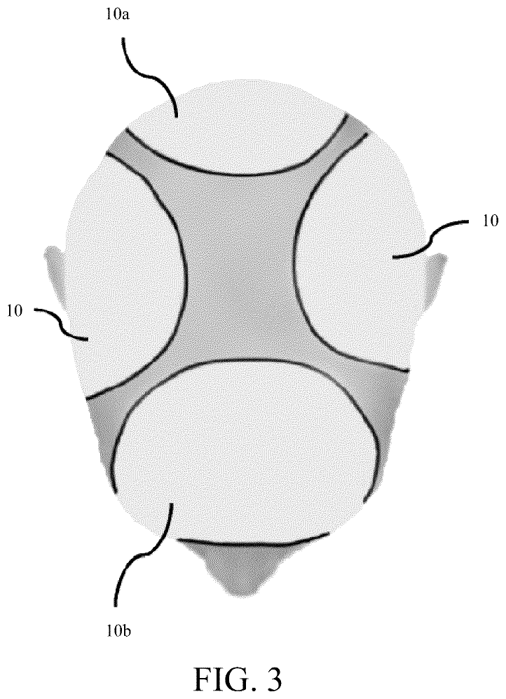

Referring to FIG. 3, a method of applying tumor treating fields to a subject's body can comprise positioning a first transducer apparatus 10a, wherein transducer apparatus 10a is in accordance with embodiments disclosed herein, in a first position at a first location of the subject's body. For example, the first location can be on the head of the subject. In other aspects, the first location can be on the torso, or anywhere else on the subject's body. An electric field can be induced between the first transducer apparatus 10a and a second transducer apparatus 10b located at a second location of the subject's body. In some aspects, the second transducer apparatus can be a transducer apparatus as described herein. In other aspects, the second transducer apparatus 10b can be any suitable transducer apparatus that, when cooperating with the first transducer apparatus 10a, provides TTFields to a target region of the body of the subject.

In some aspects, and for each of the embodiments described herein, the transducer apparatus further comprises an anisotropic material layer on the front side of the electrode elements and electrically coupled to the electrode elements. The anisotropic material layer may have anisotropic thermal properties and/or anisotropic electrical properties. If the anisotropic material layer has anisotropic thermal properties (for example, greater thermal conductivity in the plane of the layer than through the plane of the layer), then the layer spreads the heat out more evenly over a larger surface area. The thermal conductivity in the plane is more than ten times higher than the first thermal conductivity.

For example, it may be more than: 1.5 times, 2 times, 3 times, 5 times, 10 times, 20 times, 100 times, 200 times, or even more than 1,000 times higher in the plane of the layer than through the plane of the layer. If the anisotropic material layer has anisotropic electrical properties (for example, greater electrical conductivity, or, conversely, lower resistance, in the plane of the layer than through the plane of the layer), then the layer spreads the current out more evenly over a larger surface area. For example, the resistance of the anisotropic material layer in the plane may be less than half of the resistance through the plane, or it may be less than 10%. For example, the resistance of the anisotropic material layer in the plane of the layer may be less than: 75%, 50%, 40%, 30%, 20%, 10%, 5%, 1%, 0.5%, or even less than 0.1% of the resistance through the plane of the layer.

In some embodiments, the anisotropic material layer is anisotropic with respect to electrical conductivity properties. In some embodiments, the anisotropic material layer is anisotropic with respect to thermal conductivity properties. In some preferred embodiments, the anisotropic material layer is anisotropic with respect to both electrical conductivity properties and thermal conductivity properties. In some embodiments (e.g., when the anisotropic material layer is a sheet of pyrolytic graphite), the anisotropic material layer has both anisotropic electrical properties and anisotropic thermal properties.

The anisotropic material layer may comprise graphite (e.g., a sheet of graphite or a graphite sheet). Examples of suitable forms of graphite include synthetic graphite, such as pyrolytic graphite (e.g., Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied as MinGraph® 2010A Flexible Graphite, available from Mineral Seal Corp., Tucson, Arizona, USA), or graphitized polymer film, e.g., graphitized polyimide film, (including, but not limited to, that supplied by Kaneka Corp., Moka, Tochigi, Japan). Accordingly, for any of the embodiments described herein, the anisotropic material layer on the front side of the electrode elements may comprise graphite. Further, for any of the embodiments described herein, the anisotropic material layer on the front side of the electrode elements may comprise pyrolytic graphite, graphite foil made from compressed high purity exfoliated mineral graphite, or graphitized polymer film. In alternative embodiments, conductive anisotropic materials other than graphite may be used instead of graphite.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.
Aspect 1: A transducer apparatus for delivering tumor treating fields to a subject's body, the transducer apparatus comprising:
   an array of electrode elements arranged around a centroid and each electrode element equidistant or approximately equidistant from the centroid; and
   a PCB or flex circuit comprising at least one electrical connector portion, wherein each respective electrical connector portion of the at least one electrical connector portion extends between a corresponding pair of adjacent electrode elements of the array of electrode elements, wherein each electrode element of the array of electrode elements comprises a portion most proximal to the centroid of the array, and wherein each electrical connector portion is spaced radially outwardly of the portions of the corresponding pair of adjacent electrode elements most proximal to the centroid of the array.
Aspect 2: The transducer apparatus of aspect 1, wherein each electrode element of the array of electrode elements extends radially outward from the centroid and defines a taper toward the centroid.
Aspect 3: The transducer apparatus of aspect 2, wherein the portion of each electrode element most proximal to the centroid of the array of electrode elements is spaced from the centroid by a first radius, wherein each electrical connector portion is spaced from the centroid by a radius that is at least 15% greater than the first radius.
Aspect 4: The transducer apparatus of aspect 2 or aspect 3, wherein each electrode element of the array of electrode elements has an end proximal to the centroid and an end distal to the centroid, and a radial dimension measured between the end proximal to the centroid and the end distal to the centroid, wherein each electrical connector portion of the at least one electrical connector portion connects to each respective electrode element at a location positioned between 10% and 90% of the radial dimension along the radial dimension of each electrode element of a respective pair of adjacent electrode elements, wherein the location is measured as a percentage along the radial dimension of the electrode element proceeding from the end proximal to the centroid to the end distal to the centroid.
Aspect 5: The transducer apparatus of aspect 4, wherein each electrical connector portion of the at least one electrical connector portion connects to each respective electrode element at a location positioned between 10% and 50% of the radial dimension along the radial dimension of each electrode element of the respective pair of adjacent electrode elements.
Aspect 6: The transducer apparatus of any one of aspects 2-5, wherein the at least one electrical connector portion comprises a plurality of electrical connector portions, wherein the plurality of electrical connector portions are arranged in a circular, substantially circular, oval, or substantially oval profile about the centroid.
Aspect 7: The transducer apparatus of any one of aspects 2-6, wherein the array of electrode elements comprises six electrode elements approximately circumferentially equally spaced about the centroid, wherein the six electrode elements are coupled by one of:
   five electrical connector portions extending between adjacent pairs of the six electrode elements; or
   four electrical connector portions extending between adjacent pairs of the six electrode elements and a bridging connector extending between two of the six electrode elements, wherein the bridging connector is further configured to couple to an AC current source.
Aspect 8: The transducer apparatus of any one of aspects 2-7, wherein the array of electrode elements comprises six electrode elements each having a first shape and a first area, wherein adjacent pairs of electrode elements of the six electrode elements are spaced from each other by a void space having an area approximately equal to the first area.
Aspect 9: The transducer apparatus of any one of aspects 2-8, wherein the array of electrode elements has from two electrodes to six electrode elements.
Aspect 10: The transducer apparatus of any one of the preceding aspects, wherein each electrode element of the array of electrode elements has a front face facing the subject's body and a front side, and the transducer apparatus further comprises an anisotropic material layer on the front side of each electrode element of the array of electrode elements and electrically coupled to the electrode elements.
Aspect 11: The transducer apparatus of any one of the preceding aspects, wherein each electrode element of the array of electrode elements has a front face facing the subject's body and a front side, and the transducer apparatus further comprises a layer of dielectric material on the front face of each electrode element of the array of electrode elements.
Aspect 12: The transducer apparatus of any one of the preceding aspects, wherein the electrode elements are coupled to the PCB or flex circuit.
Aspect 13: A transducer apparatus for delivering tumor treating fields to a subject's body, the transducer apparatus comprising:
   a plurality of electrode elements having a centroid and arranged in a circular or oval region around the centroid, wherein the plurality of electrode elements comprise at least a first electrode element and a second electrode element;
   a printed circuit board (PCB) or flex circuit comprising a plurality of electrical connector portions;
   wherein each electrode element of the plurality of electrode elements is connected to at least one adjacent electrode element in the circular or oval region by a respective electrical connector portion of the plurality of electrical connector portions, wherein at least one electrical connector portion of the plurality of electrical connector portions has an alternating-profile extending between the first and second electrode elements.
Aspect 14: The transducer apparatus of aspect 13, wherein the alternating-profile is undulating, serpentine, wavelike, or zig-zagged.
Aspect 15: The transducer apparatus of aspect 13 or aspect 14, wherein the electrode elements of the plurality of electrode elements are equidistant or approximately equidistant from the centroid.
Aspect 16: The transducer apparatus of any one of aspects 13-15, wherein each electrode element of the plurality of electrode elements defines a taper toward the centroid. The transducer apparatus of aspect 1, wherein each respective electrical connector portion of the plurality of electrical connector portions extends between a corresponding pair of adjacent electrode elements of the array of electrode elements.
Aspect 17: The transducer apparatus of any one of aspects 13-16, wherein each electrode element of the array of electrode elements extends radially outwardly from the centroid, wherein each electrode element of the array of electrode elements comprises a portion most proximal to the centroid of the array, wherein each electrical connector portion of the PCB or flex circuit extends between a corresponding pair of adjacent electrode elements of the array of electrode elements and couples adjacent electrode elements at the respective portions of the electrode elements most proximal to the centroid of the array.
Aspect 18: The transducer apparatus of any one of aspects 13-17, wherein each electrode element of the array of electrode elements extends radially outwardly from the centroid, wherein each electrode element of the array of electrode elements comprises a portion most proximal to the centroid of the array, wherein each electrical connector portion of the PCB or flex circuit extends between a corresponding pair of adjacent electrode elements of the array of electrode elements and is spaced radially outwardly of the portions of the corresponding pair of electrode elements most proximal to the centroid of the array.
Aspect 19: The transducer apparatus of aspect 18, wherein the plurality of electrical connector portions are arranged in a substantially circular or oval profile about the centroid.
Aspect 20: The transducer apparatus of aspect 18, wherein the portion of each electrode element most proximal to the centroid of the array of electrode elements is spaced from the centroid by a first radius, wherein each electrical connector portion is spaced from the centroid by a radius that is at least 20% greater than the first radius.
Aspect 21: The transducer apparatus of aspect 18, wherein each electrode element of the array of electrode elements has a radial dimension, wherein each electrical connector portion of the plurality of electrical connector portions connects to each respective electrode element at a location positioned between 10% and 90% of the radial dimension along the radial dimension of each electrode element of the respective pair of electrode elements, wherein the location is measured as a percentage along the radial dimension of the electrode element proceeding from the end proximal to the centroid to the end distal to the centroid.
Aspect 22: The transducer apparatus of any one of the preceding aspects, wherein the electrode elements are coupled to the PCB or flex circuit.
Aspect 23: The transducer apparatus of any one of the preceding aspects, wherein the PCB or flex circuit at least partially forms the electrode elements.
Aspect 24: The transducer apparatus of any one aspects 13-23, wherein each electrode element of the array of electrode elements has a front face facing the subject's body and a front side, and the transducer apparatus further comprises an anisotropic material layer on the front side of each electrode element of the array of electrode elements and electrically coupled to the electrode elements.
Aspect 25: The transducer apparatus of any one aspects 13-24, wherein each electrode element of the array of electrode elements has a front face facing the subject's body and a front side, and the transducer apparatus further comprises a layer of dielectric material on the front face of each electrode element of the array of electrode elements.
Aspect 26: The transducer apparatus of any one aspects 13-25, wherein the electrode elements are coupled to the PCB or flex circuit.
Aspect 27: The transducer apparatus of claim 2, wherein the array of electrode elements comprises three electrode elements approximately circumferentially equally spaced about the centroid, wherein the three electrode elements are coupled by one of:
   two electrical connector portions extending between adjacent pairs of the three electrode elements; or
   one electrical connector portion extending between a first adjacent pair of the three electrode elements and a bridging connector extending between a second adjacent pair of the three electrode elements, wherein the bridging connector is further configured to couple to an AC current source.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
an array of electrode elements arranged around a centroid and each electrode element equidistant or approximately equidistant from the centroid; and
a PCB or flex circuit comprising at least one electrical connector portion, wherein each respective electrical connector portion of the at least one electrical connector portion extends between a corresponding pair of adjacent electrode elements of the array of electrode elements, wherein each electrode element of the array of electrode elements comprises a portion most proximal to the centroid of the array, and wherein each electrical connector portion is spaced radially outwardly of the portions of the corresponding pair of adjacent electrode elements most proximal to the centroid of the array.

2. The transducer apparatus of claim 1, wherein each electrode element of the array of electrode elements extends radially outward from the centroid and defines a taper toward the centroid.

3. The transducer apparatus of claim 2, wherein the portion of each electrode element most proximal to the centroid of the array of electrode elements is spaced from the centroid by a first radius, wherein each electrical connector portion is spaced from the centroid by a radius that is at least 15% greater than the first radius.

4. The transducer apparatus of claim 2, wherein each electrode element of the array of electrode elements has an end proximal to the centroid and an end distal to the centroid, and a radial dimension measured between the end proximal to the centroid and the end distal to the centroid, wherein each electrical connector portion of the at least one electrical connector portion connects to each respective electrode element at a location positioned between 10% and 90% of the radial dimension along the radial dimension of each electrode element of a respective pair of adjacent electrode elements, wherein the location is measured as a percentage along the radial dimension of the electrode element proceeding from the end proximal to the centroid to the end distal to the centroid.

5. The transducer apparatus of claim 4, wherein each electrical connector portion of the at least one electrical connector portion connects to each respective electrode element at a location positioned between 10% and 50% of the radial dimension along the radial dimension of each electrode element of the respective pair of adjacent electrode elements.

6. The transducer apparatus of claim 2, wherein the at least one electrical connector portion comprises a plurality of electrical connector portions, wherein the plurality of electrical connector portions are arranged in a circular, substantially circular, oval, or substantially oval profile about the centroid.

7. The transducer apparatus of claim 2, wherein the array of electrode elements comprises six electrode elements approximately circumferentially equally spaced about the centroid, wherein the six electrode elements are coupled by one of:
five electrical connector portions extending between adjacent pairs of the six electrode elements; or
four electrical connector portions extending between adjacent pairs of the six electrode elements and a bridging connector extending between two of the six electrode elements, wherein the bridging connector is further configured to couple to an AC current source.

8. The transducer apparatus of claim 2, wherein the array of electrode elements comprises six electrode elements each having a first shape and a first area, wherein adjacent pairs of electrode elements of the six electrode elements are spaced from each other by a void space having an area approximately equal to the first area.

9. The transducer apparatus of claim 2, wherein the array of electrode elements has from two electrodes to six electrode elements.

10. The transducer apparatus of claim 1, wherein each electrode element of the array of electrode elements has a front face facing the subject's body and a front side, and the transducer apparatus further comprises an anisotropic material layer on the front side of each electrode element of the array of electrode elements and electrically coupled to the electrode elements.

11. The transducer apparatus of claim 1, wherein each electrode element of the array of electrode elements has a front face facing the subject's body and a front side, and the transducer apparatus further comprises a layer of dielectric material on the front face of each electrode element of the array of electrode elements.

12. The transducer apparatus of claim 1, wherein the electrode elements are coupled to the PCB or flex circuit.

13. A transducer apparatus for delivering tumor treating fields to a subject's body, the transducer apparatus comprising:
a plurality of electrode elements having a centroid and arranged in a circular or oval region around the centroid, wherein the plurality of electrode elements comprise at least a first electrode element and a second electrode element;
a printed circuit board (PCB) or flex circuit comprising a plurality of electrical connector portions;
wherein each electrode element of the plurality of electrode elements is connected to at least one adjacent electrode element in the circular or oval region by a respective electrical connector portion of the plurality of electrical connector portions, wherein at least one electrical connector portion of the plurality of electrical connector portions has an alternating-profile extending between the first and second electrode elements.

14. The transducer apparatus of claim 13, wherein the alternating-profile is undulating, serpentine, wavelike, or zig-zagged.

15. The transducer apparatus of claim 13, wherein the electrode elements of the plurality of electrode elements are equidistant or approximately equidistant from the centroid.

16. The transducer apparatus of claim 13, wherein each electrode element of the plurality of electrode elements defines a taper toward the centroid.

17. The transducer apparatus of claim 13, wherein each electrode element of the array of electrode elements extends radially outwardly from the centroid, wherein each electrode element of the array of electrode elements comprises a portion most proximal to the centroid of the array, wherein each electrical connector portion of the PCB or flex circuit extends between a corresponding pair of adjacent electrode elements of the array of electrode elements and couples adjacent electrode elements at the respective portions of the electrode elements most proximal to the centroid of the array.

18. The transducer apparatus of claim 13, wherein each electrode element of the array of electrode elements extends radially outwardly from the centroid, wherein each electrode element of the array of electrode elements comprises a portion most proximal to the centroid of the array, wherein each electrical connector portion of the PCB or flex circuit extends between a corresponding pair of adjacent electrode elements of the array of electrode elements and is spaced radially outwardly of the portions of the corresponding pair of electrode elements most proximal to the centroid of the array.

19. The transducer apparatus of claim 18, wherein the portion of each electrode element most proximal to the centroid of the array of electrode elements is spaced from the centroid by a first radius, wherein each electrical connector portion is spaced from the centroid by a radius that is at least 20% greater than the first radius.

20. The transducer apparatus of claim 18, wherein each electrode element of the array of electrode elements has a radial dimension, wherein each electrical connector portion of the plurality of electrical connector portions connects to each respective electrode element at a location positioned between 10% and 90% of the radial dimension along the radial dimension of each electrode element of the respective pair of electrode elements, wherein the location is measured as a percentage along the radial dimension of the electrode element proceeding from the end proximal to the centroid to the end distal to the centroid.
